(19) United States
(12) Patent Application Publication
(10) Pub. No.: US 2025/0049425 A1
(43) Pub. Date: Feb. 13, 2025

(54) VASCULAR CLOSURE DEVICE WITH RETRACTION ELEMENT FOR REPOSITIONING A SEALING PLUG

(71) Applicant: Teleflex Life Sciences LLC, Wilmington, DE (US)

(72) Inventors: Joshua Brenizer, Oak Grove, MN (US); Darren Prom, Coon Rapids, MN (US); Christopher E. Buller, Toronto (CA); Nicholas Mark Donnay, Plymouth, MN (US)

(73) Assignee: Teleflex Life Sciences LLC, Wilmington, DE (US)

(21) Appl. No.: 18/797,456

(22) Filed: Aug. 7, 2024

Related U.S. Application Data
(60) Provisional application No. 63/518,043, filed on Aug. 7, 2023.

(51) Int. Cl. A61B 17/00

(52) U.S. Cl. CPC A61B 17/0057; A61B 2017/00004; A61B 2017/00575; A61B 2017/00615; A61B 2017/00619; A61B 2017/00623

(57) ABSTRACT

A vascular closure device is configured to seal a puncture in an artery or vein. The vascular closure device includes a deployment assembly having a proximal end and a distal end opposite the proximal end, a suture carried by the deployment assembly, and a sealing plug carried by the deployment assembly and coupled to the suture. The sealing plug is configured to exit the distal end of the deployment assembly for deployment in the puncture. The vascular closure device includes a retraction element coupled to the sealing plug, the retraction element configured to, after deployment of the sealing plug, retract the sealing plug in a proximal direction.

### CROSS-REFERENCE TO RELATED APPLICATIONS

This application claims the benefit of priority under 35 U.S.C. § 119 (c) to U.S. Provisional Appln. Ser. No. 63/518,043, filed Aug. 7, 2023, which is incorporated herein by reference in its entirety.

### TECHNICAL FIELD

The present application is directed to a vascular closure device with retraction element for repositioning a sealing plug.

### BACKGROUND

Percutaneous access of the vascular system for vascular device delivery is a common medical procedure. Typically, this involves using a hollow needle to puncture a vessel, then introducing an introducer sheath to open the puncture site for the introduction of catheters and wire guides for navigation through the vascular system to facilitate delivery. For example, in many cases, vascular access requires introduction of catheters and wire guides through the femoral artery. Once the procedure is completed, the devices are removed from the patient and pressure is applied to the puncture site to stop the bleeding. Thereafter, the puncture may be sealed using a closure device.

Closure devices generally consist of three basic sealing components: a footplate (or anchor) member, a sealing member (or plug), and a filament (or suture). To lock the components together within the puncture, a locking member may be used.

### SUMMARY

An embodiment of the present disclosure includes a vascular closure device configured to seal a puncture in an artery or vein. The vascular closure device may include a deployment assembly. The vascular closure device may further include a suture carried by the deployment assembly. The vascular closure device may further include a sealing plug movably coupled to the suture. The sealing plug may have a forward end, a trailing end, and sides that extend from the forward end toward the trailing end. The sealing plug may be carried by the deployment assembly in a first configuration and may be located outside of the deployment assembly in a second configuration. The vascular closure device may further include a retraction clement positioned on the sealing plug. The vascular closure device may further include a tether coupled to the retraction element. When the sealing plug is in the second configuration located outside of the deployment assembly, tension applied to the tether may pull the retraction element in a proximal direction to move the sealing plug in the proximal direction.

An embodiment of the present disclosure includes a vascular closure device configured to seal a puncture in an artery or vein. The vascular closure device may include a suture. The vascular closure device may further include a footplate coupled to the suture, where the footplate is configured for deployment in a lumen of the artery or vein. The vascular closure device may further include a sealing plug movably coupled to the suture, the sealing plug having a forward end, a trailing end, and sides that extend from the forward end toward the trailing end. The vascular closure device may further include a retraction element positioned at the sealing plug. The vascular closure device may further include a tether coupled to the retraction element.

An embodiment of the present disclosure includes a vascular closure device configured to seal a puncture in an artery or vein. The vascular closure device may include a deployment assembly having a proximal end and a distal end opposite the proximal end. The vascular closure device may further include a suture carried by the deployment assembly. The vascular closure device may further include a footplate carried by the deployment assembly and coupled to the suture. The footplate may be configured to exit the distal end of the deployment assembly for deployment in the puncture. The vascular closure device may further include a scaling plug movably coupled to the suture, the sealing plug having a forward end, a trailing end, and sides that extend from the forward end toward the trailing end. The sealing plug may be carried by the deployment assembly in a first configuration and may be located outside of the deployment assembly in a second configuration. The vascular closure device may further include a retraction element positioned at the sealing plug. The vascular closure device may further include a tether coupled to the retraction element. According to such embodiments, when the sealing plug is in the second configuration located outside of the deployment assembly, tension applied to the tether may pull the retraction element in a proximal direction to move the sealing plug in the proximal direction.

### BRIEF DESCRIPTION OF THE DRAWINGS

The foregoing summary, as well as the following detailed description of example embodiments of the disclosure, will be better understood when read in conjunction with the appended drawings, in which there is shown example embodiments for the purposes of illustration. It should be understood, however, that the application is not limited to the precise arrangements and systems shown in the drawings, in which:

FIG. 1 is a perspective view of a vascular closure device in accordance with an embodiment of the present disclosure;

FIG. 2 is a partial cut-away view of the vascular closure device shown in FIG. 1;

FIG. 3 is a front view of a sealing device according to an embodiment of the present disclosure and associated with the vascular closure device in FIG. 1 where the sealing plug is in a compressed state;

FIG. 4 is a side view of the sealing device shown in FIG. 3 where the sealing plug is in a non-compressed state;

FIG. 5 is a top view of the retraction element shown in FIGS. 3-4;

FIG. 6 is side view of the retraction element shown in FIG. 5;

FIG. 7 is a partial sectional view showing the sealing device shown in FIGS. 3-6 disposed in a distal end of the vascular closure device shown in FIGS. 1-2;

FIG. 8 is a schematic perspective view of a guide member for the vascular closure device shown in FIG. 1.

FIG. 9 is a front view of a sealing device according to an embodiment of the present disclosure and associated with the vascular closure device in FIG. 1 where the sealing plug is in a non-compressed state;

FIG. 10 is a front view of the sealing device shown in FIG. 9 where the sealing plug is in a compressed state;

FIG. 11 is a side view of the retraction element shown in FIGS. 9-10;

FIG. 12 is a top view of the retraction element shown in FIG. 11;

FIG. 13 is a partial sectional view showing the sealing device shown in FIGS. 9-12 disposed in a distal end of the vascular closure device shown in FIGS. 1-2;

FIG. 14 is a schematic showing an access sheath partially disposed within a vessel through a puncture site in the vessel;

FIG. 15 is a schematic showing the closure device of FIG. 1 translated into an access channel of the access sheath such that a distal end of the footplate is positioned distal to a distal end of the access sheath and over a guidewire;

FIG. 16 is a schematic showing positioning of the closure device of FIG. 1 relative to the anatomy;

FIG. 17 is a schematic showing actuation of the actuator to release the footplate;

FIG. 18 is a schematic showing the deployment assembly being pulled in a proximal direction such that the footplate abuts the vessel wall;

FIG. 19 is a schematic showing deployment of a plug of the closure device;

FIG. 20 is a schematic showing actuation of the retraction element via a tether to reposition a sealing plug of the closure device;

FIG. 21 is a schematic showing deployment of a locking member against the plug;

FIG. 22 is a schematic showing the locking member being tamped against the plug with a tamper; and

FIG. 23 is a schematic showing the deployment of the sealing device fully sealing the puncture site.

### DETAILED DESCRIPTION OF ILLUSTRATIVE EMBODIMENTS

Certain terminology is used in the following description for convenience only and is not limiting. The words “right”, “left”, “lower” and “upper” designate directions in the drawings to which reference is made. The words “proximally” and “distally” refer to directions toward and away from, respectively, the individual operating the system. The terminology includes the above-listed words, derivatives thereof and words of similar import.

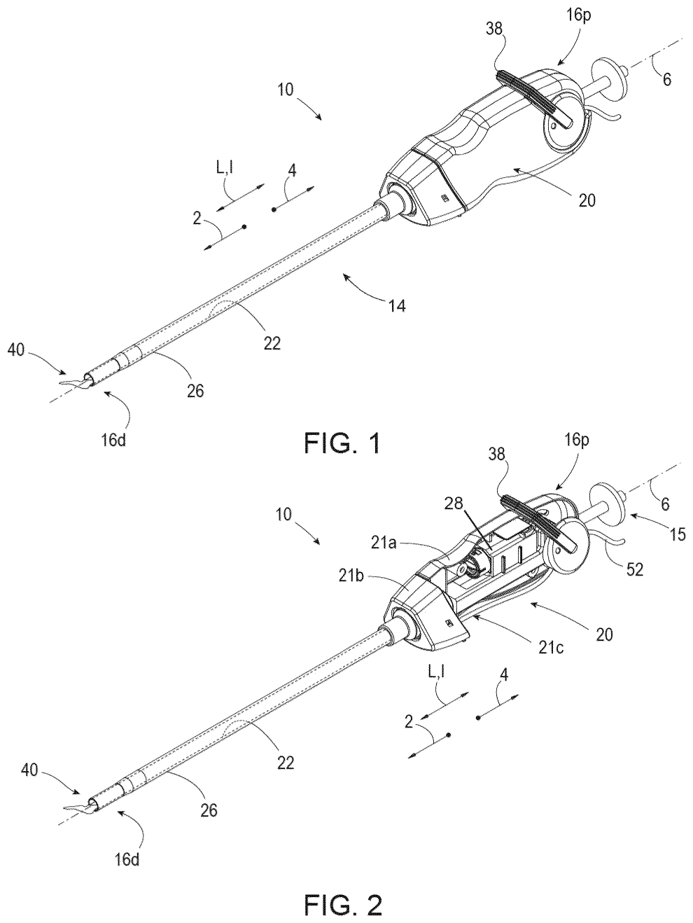

Referring to FIGS. 1-13, embodiments of a vascular closure device 10 may include a scaling unit 18 at least partially disposed within a deployment assembly 14, and a retraction clement 50. The vascular closure device 10 can be configured such that after the deployment assembly 14 is inserted into a vessel through a puncture site of the vessel, the scaling unit 18 is deployed to thereby seal or otherwise close the puncture site of the vessel. After deployment but before the sealing unit 18 is in its final sealing configuration, a user can retract a scaling plug 88 of the sealing unit 18 in a proximal direction, and even into the deployment assembly 14, with the retraction assembly 50. The retraction assembly 50, in particular, may allow the user to withdraw or pull back the sealing plug 88 in the event of incorrect positioning or placement of the sealing plug 88 during a medical procedure.

The deployment assembly 14 may be configured to control the orientation of a footplate 40 of the sealing unit 18 during use. In accordance with the illustrated embodiment, the deployment assembly 14 may include a release component 22 (shown in dashed lines in FIGS. 1 and 2) that restrains the footplate 40, a delivery component 26 that contains at least a portion of the footplate 40 and a suture 44 of the sealing unit 18, and one or more actuators, such as deployment actuator 38. The release component 22 may be operatively coupled or associated with the suture 44 such that actuation of the deployment actuator 38 causes the release component 22 to 1) release the footplate 40, and 2) apply tension to the suture 44, which urges the footplate 40 against the delivery component 26 and orients the footplate 40 in the sealing position.

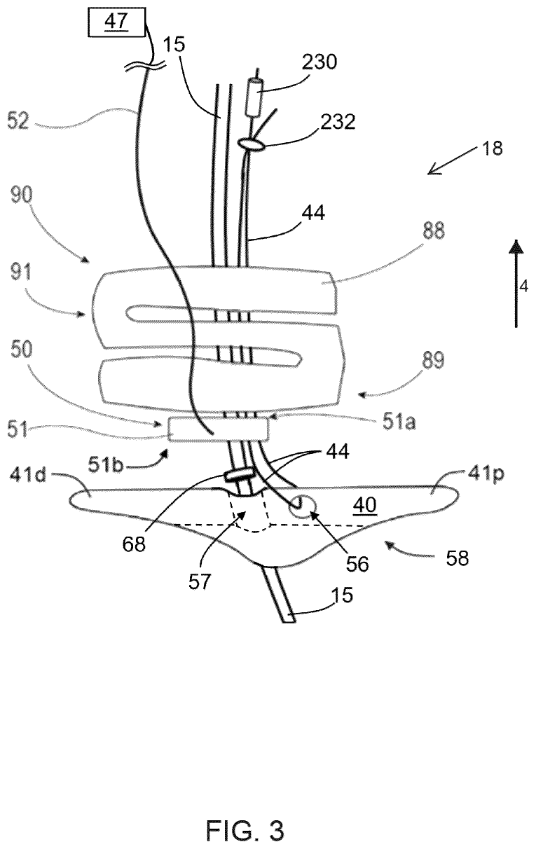

Turning to FIGS. 3-7, the sealing unit 18 may include the footplate 40 connected to the suture 44, a plug 88 coupled to the suture 44 and spaced from the footplate 40 in a proximal direction 4, and a locking member 230 on the suture 44, proximal to the plug 88.

The footplate 40 may include or define a distal end 41d and a proximal end 41p opposed to the distal end 41d, a first set of apertures 56 configured to receive the suture 44, a guide member aperture 57, and an engagement member, portion, and/or feature 58. The suture 44 may extend through the first set of apertures 56 as illustrated, such that an end of the suture 44 may be formed into a slidable knot 232. The knot 232 may be slidable along the suture 44 between the plug 88 and the locking member 230. The guide member 15 may extend through the aperture 57. In an implanted state, the footplate 40 may be positioned adjacent to an inner surface of the vessel, and the locking member 230 may squeeze the footplate 40 and the plug 88 against the outer surface vessel to seal the puncture. The guide member 15 may extend through the sealing unit 18 and may be configured to receive a guidewire 150, as will be discussed below.

The sealing unit 18 may be formed with materials suitable for surgical procedures. For instance, the footplate 40 can be made of any biocompatible material, non-limiting examples of which may include or comprise a polylactic-coglycolic acid or other synthetic absorbable polymer that degrades in the presence of water into naturally occurring metabolites. In some embodiments, the footplate can be made of stainless steel, biocorrodible iron, and/or biocorrodible magnesium. It should be appreciated, however, that the footplate 40 can be made of other materials and can have other configurations so long as it can be seated inside the vessel against the vessel wall.

The sealing plug 88 can comprise a strip of compressible, resorbable, collagen foam and can be made of a fibrous collagen mix of insoluble and soluble collagen that may be cross-linked for strength. In the illustrated embodiment, the scaling plug 88 has a forward end 89, a trailing end 90, and sides 91 that extend from the forward end 89 toward the trailing end 90. It should be appreciated, however, that the sealing plug 88 can have any configuration as desired and can be made from any material as desired. The suture 44 can be any elongate member, such as, for example a filament, wire, thread, or braid. The sealing plug 88 may be movably coupled to the suture 44.

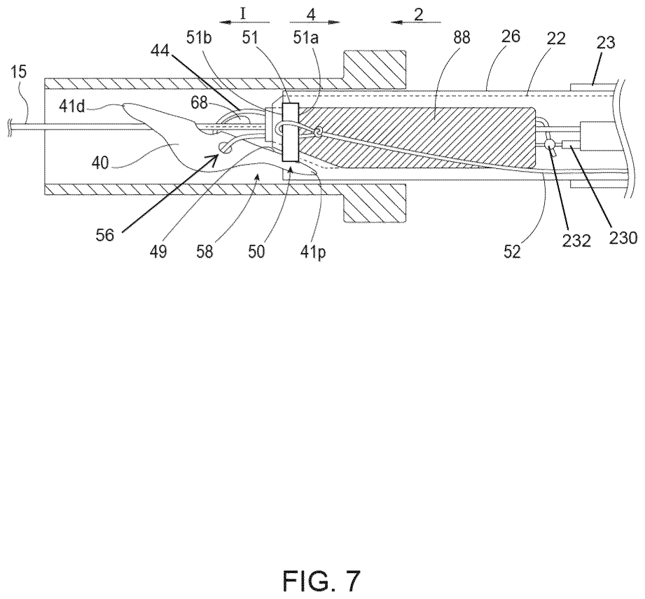

The deployment assembly 14, including the release component 22 and delivery component 26, may be constructed and may function in accordance with one or more of the devices disclosed in U.S. Pat. No. 11,364,024, the entire contents of which are incorporated by reference into the present application. Referring again to FIGS. 1, 2, and 7, the deployment assembly 14 may be elongate along a longitudinal direction L and may include a proximal end 16p and a distal end 16d spaced from the proximal end 16p along an axis 6 that may be aligned with the longitudinal direction L. The longitudinal direction L can include and define a distal direction 2 that extends from the proximal end 16p toward the distal end 16d. Further, the longitudinal direction L can include and define a proximal direction 4 that is opposite the distal direction 2 and that extends from distal end 16d toward the proximal end 16p. The deployment assembly 14 may be configured to insert the footplate 40 into the vessel along an insertion direction I (see FIG. 7). The longitudinal direction L can be aligned with the insertion direction I during a portion of the sealing procedure.

Turning to FIGS. 1 and 2, in accordance with the illustrated embodiment, the deployment assembly 14 may include a handle member 20, the release component 22 supported by the handle member 20, the delivery component 26, a tensioner 28 supported by the handle member 20, and at least one deployment actuator 38. A portion of release component 22 is shown in dashed lines in FIGS. 1 and 2.

The deployment actuator 38 may be coupled to both the handle member 20 and the release component 22. The deployment actuator 38 may be configured to 1) cause the release component 22 to move in the proximal direction 4 from a first or initial position relative to the delivery component 26 into a second or releasing position relative to the delivery component 26, and 2) apply a tensile force to the suture 44 during or subsequent to movement of the release component 22 from the initial position into the release position. The description below refers to the release component 22 being movable relative to the delivery component 26, but the deployment assembly 14 can be configured so that the delivery component 26 is movable relative to the release component 22 in some examples. The deployment assembly 14 may also include the guide member 15 that extends through the deployment assembly 14, and an optional outer sheath 23 (see FIG. 7) that contains and supports portions of the release component 22 and delivery component 26.

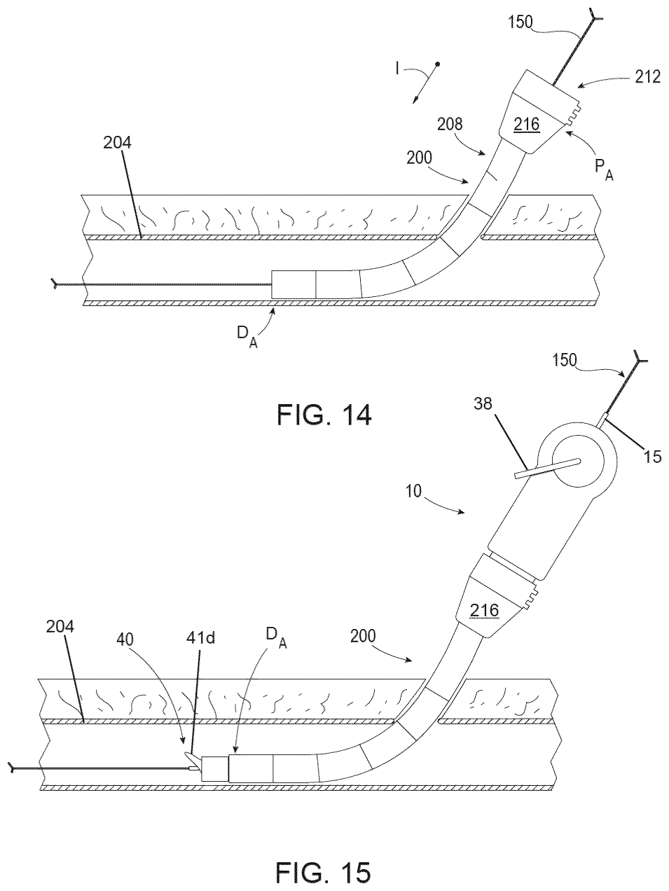

Continuing with FIGS. 1 and 2, the handle member 20 may include a housing 21a, a sheath hub 21b, and a cavity 21c defined at least partly by housing 21a and sheath hub 21b. The cavity 21c may be sized to contain a portion of the release and delivery components 22 and 26, along with the tensioner 28. The sheath hub 21b may be configured to mate with an access sheath 208 (See FIGS. 6 and 14).

The release component 22 may be elongate along a first or longitudinal direction L and may define a distal end and a proximal end spaced from the distal end along the longitudinal direction L. The release component 22 may include a release tube body that is elongate along the longitudinal direction L. The release tube body may define a release tube channel that extends along the longitudinal direction L from the hub toward the proximal end. The release tube channel may be sized to slidably receive a portion of the delivery component 26 such that the release component 22 is movable relative to the delivery component 26. Non-limiting examples of one or more of these features and components thereof, including additional features of the release component 22, are described in U.S. Pat. No. 11,364,024.

The tensioner 28 may be positioned in the handle member 20 and coupled to the proximal end of the release component 22. In one example, the suture 44 may extend around a pulley and into the tensioner 28. As the release component 22 is pulled in the proximal direction 4, the pulley may pull the suture 44 in proximal direction 4, thereby applying a tensile force to the footplate 40. Other arrangements may be used to apply to tension to the suture 44 and footplate 40 as needed.

The delivery component 26 may be coupled to the tensioner 28 and may extend along the release component 22 toward the distal end 16d of the deployment assembly 14. The delivery component 26 may include a delivery tube body that is elongate along the first direction L and that defines a distal end and a proximal end spaced from the distal end in the first direction L. The delivery tube body may define a delivery tube channel that extends at least partially through the delivery tube body along the first direction L. As illustrated, the proximal end of delivery component may be fixed to the tensioner 28, and the distal end of the delivery component may be configured to hold at least a portion of the sealing unit 18 (see FIG. 7). Non-limiting examples of one or more of these features and components thereof, including additional features of the release component 26, are described in U.S. Pat. No. 11,364,024.

The delivery tube channel may be sized to retain at least a portion of the sealing unit 18. In particular, the plug 88 and locking member 230 may be retained within the delivery tube channel, while the footplate 40 may be configured to be initially trapped between the delivery component 26 and the release component 22. For instance, the distal end of the release tube may define an offset surface 49, as shown in FIG. 7, which can be angled with respect to the longitudinal axis 6. The offset surface 49 and inner surface of the delivery component 26 may define a cavity that receives the proximal end 41p of the footplate 40 when release component 22 is in the initial position (as shown in FIG. 7). The angle of the offset surface 49 can define the orientation of the footplate 40 in this initial position, whereby the distal end 41d of the footplate 40 may be spaced some distance in the distal direction 2 beyond the distal ends of the release and delivery components 22 and 26, respectively. The suture 44 may extend from the footplate 40 through the delivery tube channel, through the proximal end around the pulley of the tensioner 28. The guide member 15 may extend through the delivery component and may exit the distal end 16d of the vascular closure device 10.

When the deployment actuator 38 is actuated, the release component 22 may move in the proximal direction 4, thereby releasing the proximal end 41p of the footplate 40 from between the release component 22 and the delivery component 26. As the release component 22 moves in the proximal direction 4, the suture 44 may be pulled in the proximal direction 4 to thereby place the suture 44 in tension and urge the footplate 40 against the distal end of the delivery component 26. At this point, the footplate 40 may be oriented in the sealing position. In the sealing position, the footplate 40 has been repositioned so that the footplate 40 is placed against the distal end of the delivery component 26 and is oriented more transversely with respect to the axis 6 compared to the position when the footplate 40 is restrained by the release component 22.

The deployment assembly 14 can include one or more actuators that are configured to transition the release component 22 into a releasing position and cause a tension to be applied to suture 44 when footplate 40 is released from the release component 22, as described above. The deployment actuator 38 may engage the release component 22 such that motion of the deployment actuator 38 relative to the handle member 20 causes the release component to translate in the proximal direction 4 and further applies a tension to the suture 44. The result may be that rotation or movement of the deployment actuator 38 causes the release component 22 to translate in the longitudinal direction L. As shown in the drawings, the deployment actuator 38 can be configured as a lever that is rotatably coupled to the handle member 20. The deployment actuator 38, however, can be a knob or a slide. It should be appreciated, however, that the deployment actuator 38 can have other configurations as desired and is not limited to the disclosed lever.

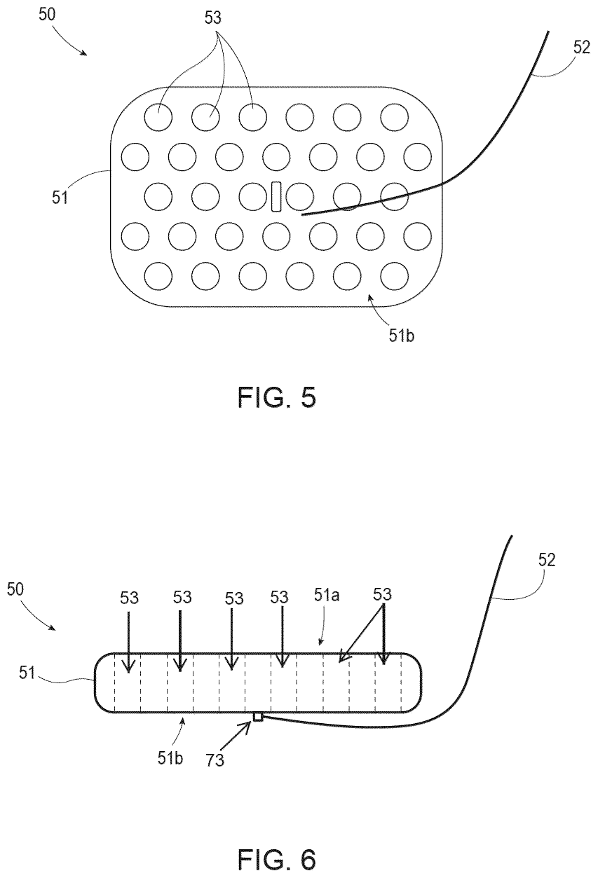

Referring to FIGS. 3-7, a sealing assembly or unit 18 having a retraction element 50 according to an embodiment of the present disclosure is described. The retraction element 50 may be configured to retract the sealing plug 88 in a proximal direction, for example in responsive to manual engagement by a user. The retraction element 50 may include a retraction body 51 having a forward surface 51a and a trailing surface 51b opposite the forward surface, wherein the trailing surface 51b is opposite a forward end 89 of the sealing plug 88. As shown in FIGS. 5 and 6, the retraction body 51 may have a plurality of apertures 53 configured to allow blood or fluid to pass through the retraction element 50. The retraction element 50 may be positioned at the forward end 89 of the sealing plug 88, where the retraction body may serve as a buttress.

The retraction element 50 (or assembly) may include a tether 52 coupled to the retraction body 51. The tether 52 may be any elongate element coupled to the retraction body 51. As such, the tether 52 may be an elongate shaft, suture, filament, wire, or rod. In the embodiment shown in FIGS. 3-7, the tether 52 extends through the deployment assembly 14. However, in alternative configurations, the tether 52 may extend alongside the deployment assembly 14. When the sealing plug 88 is in a configuration located outside of the deployment assembly 14, tension applied to the tether 52 may pull the retraction element 50 in a proximal direction to move the sealing plug 88 in the proximal direction. In some examples, the retraction element 50 may include or define a tether attachment point or portion 73, as shown for instance in FIG. 6. In addition or alternatively, the tether 52 may wrap around the retraction body 51, forming a loop or lasso, as shown in FIG. 7.

The retraction element 50 may be porous such that blood and other fluids can pass therethrough without degradation or disintegration. As a result, the retraction element 50 may be configured as a mesh structure. The retraction element 50 may also be substantially bioabsorbable. The retraction assembly may also include a retraction actuator 47 configured to pull the tether 52 in a proximal direction in order to retract at least the scaling plug 88 in a proximal direction that is opposite a distal direction. In one example, the actuator may comprise a push-pull member, which may include a rod and wire. In another example, the actuator can be a rotatable knob, rotatable lever, or a slide. In an alternative embodiment, the sealing unit 18 may include more than one retraction element, such as a first retraction element and a second retraction element.

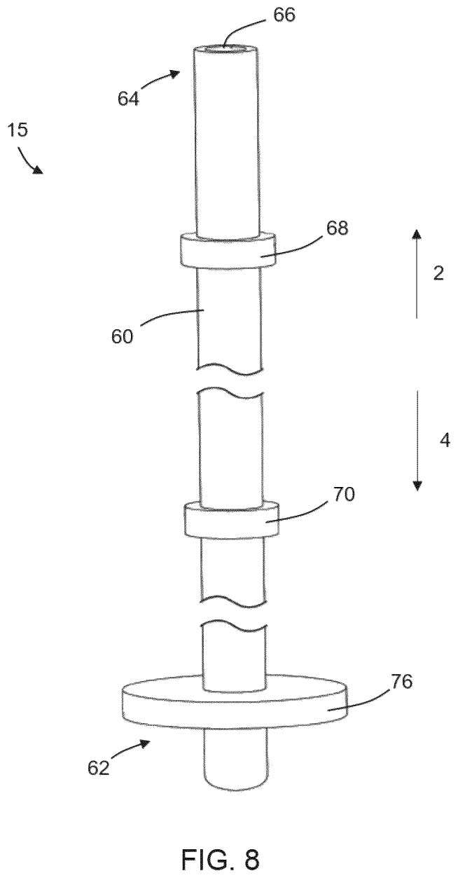

As shown in FIG. 8, the guide member 15 may have an elongate body 60 having proximal end 62, a distal end 64, and a lumen 66 that extends from the proximal end 62 to the distal end 64. The lumen 66 may be configured to receive a guidewire therethrough, such as guidewire 150 (see FIGS. 14-18). The guide member 15 may also include a footplate engagement member 68 configured to abut the footplate 40. The guide member 15 may also include an advancement engagement member 70 configured to, when a force is applied to the advancement engagement member 70, advance the guide member 15 in the distal direction. The guide member 15 may include an actuator 76, which may be configured as shown as a grip member, which in some examples may be manually engageable by a user.

In operation, the deployment assembly 14 may be initially configured to insert the footplate 40 into the vessel. When the deployment actuator 38 is actuated, the release component 22 may move in the proximal direction 4 relative to the delivery component 26 into a releasing position, thereby releasing the proximal end 41p of the footplate 40 from between the release component 22 and the delivery component 26. As the release component 22 moves in the proximal direction 4, the suture 44 may be pulled in the proximal direction 4 to thereby place the suture 44 in tension and urge the footplate 40 against the distal end of the delivery component 26. At this point, the footplate 40 may be oriented in the sealing position (see FIG. 17, for example). Accordingly, the release component 22 may be configured to restrain the footplate 40 of the sealing unit 18 during insertion of the vascular closure device 10 into the vessel and subsequently release the footplate 40 so that the footplate 40 can be oriented for the sealing procedure.

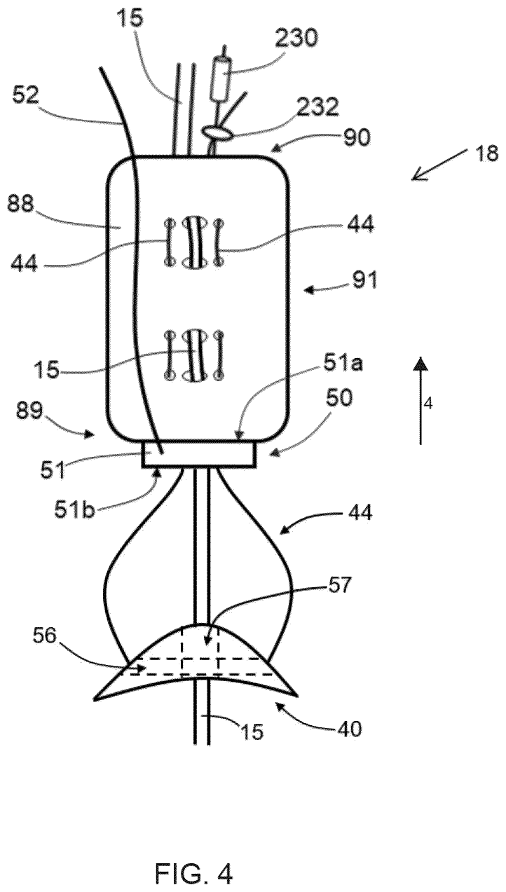

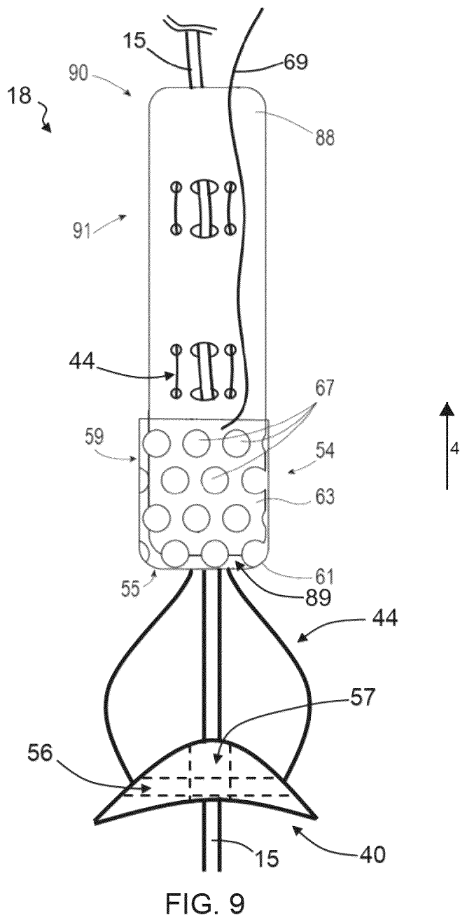

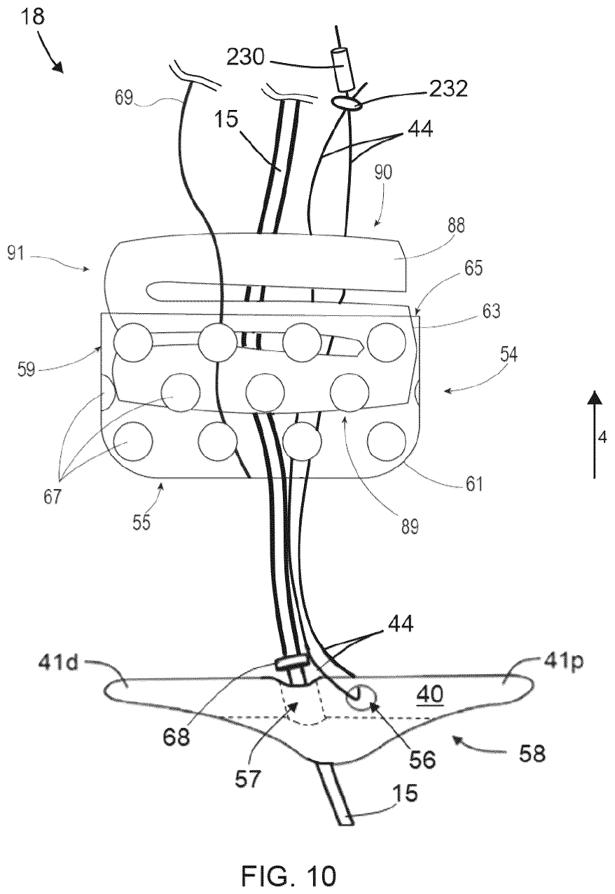

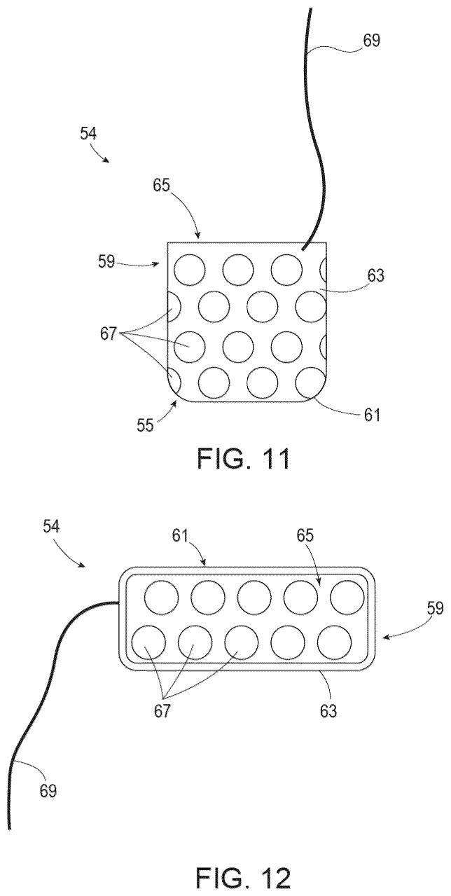

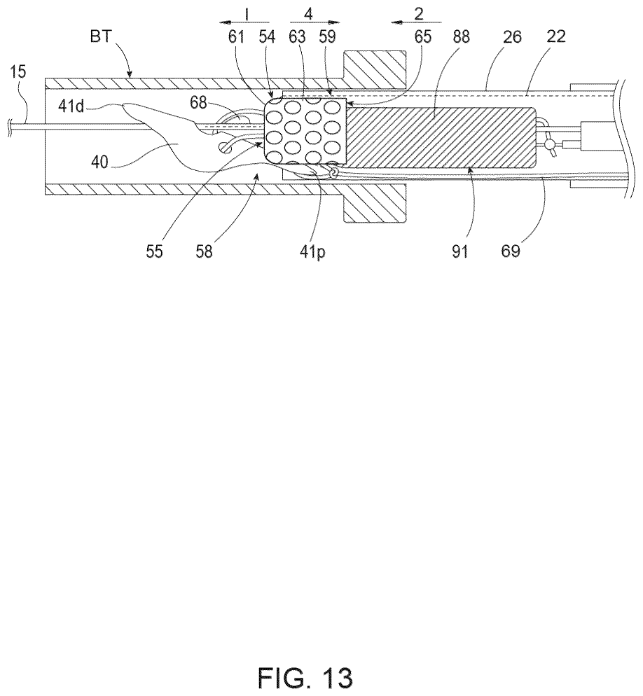

Referring to FIGS. 9-13, a sealing unit 18 having a retraction element according to another embodiment of the present disclosure is described. As shown, retraction element 54 may be configured to retract the sealing plug 88 in a proximal direction. The retraction element 54 may include a first portion 55 positioned at a forward end 89 of the scaling plug 88 and a second portion 59 adjacent to the sides 91 of the sealing plug 88. The retraction element 54 may further include a distal wall 61 and a side wall 63 that extends in a proximal direction relative to the distal end, wherein the distal wall 61 and the side wall 63 define an internal space 65 that includes or receives the forward end of the sealing plug 88. The distal wall 61 and the side wall 63 include a plurality of apertures 67 configured to allow blood or fluid to pass through the retraction element, which may be positioned at the forward end 89 of the sealing plug 88. FIG. 9 depicts the sealing unit 18 in a configuration in which the sealing plug 88 is in a non-compressed state, and FIG. 10 depicts the sealing unit 18 in another configuration in which the sealing plug 88 is in a compressed state. Components in FIGS. 9 and 10 that are similar or identical to those represented in FIGS. 3 and 4 are assigned the same numerical labels for ease of reference. FIG. 11 provides a side view of the retraction element 54, and FIG. 12 provides a top plan view of the same. FIG. 13 shows the retraction element 54 positioned within a distal portion of a vascular closure device, such as the vascular closure device 10 illustrated in FIGS. 1 and 2, as well as a proximal portion of a bypass tube (BT).

The retraction element 54 may include or be coupled with a tether 69. The tether 69 may be any elongate clement coupled to the retraction element 54. As such, the tether 69 may be an elongate shaft, suture, filament, wire, or rod. In the embodiment shown, the tether 69 extends through the deployment assembly 14. However, in alternative configurations, the tether 69 may extend alongside the deployment assembly 14. When the sealing plug 88 is in a configuration located outside of the deployment assembly 14, tension applied to the tether 69 may pull the retraction element 54 in a proximal direction to move the sealing plug 88 in the proximal direction.

The retraction element 54 may be porous such that blood and other fluids can pass therethrough without degradation or disintegration. As a result, the retraction element 54 may be configured as a mesh structure in the shape of a cup or cover that is configured to surround, slide onto and/or wrap around the distal end of the sealing plug 88. The retraction element 54 may also be substantially bioabsorbable. The retraction assembly may also include a retraction actuator (e.g., actuator 47) configured to pull the tether 69 in a proximal direction in order to retract at least the sealing plug 88 in a proximal direction that is opposite a distal direction. In one example, the actuator may include a push-pull member, which may include a rod and wire. In another example, the actuator can be a rotatable knob, rotatable lever, or a slide. In some embodiments, the sealing unit 18 may include a first retraction element and a second retraction element.

Embodiments of the present disclosure will now be described with respect to exemplary large bore procedures that utilize the vascular closure device 10 and scaling unit 18 having a retraction element 50 as disclosed in FIGS. 3-7. However, it should be noted that the sealing unit 18 having a retraction element 54 as disclosed in FIGS. 9-13 may also be utilized in large bore procedures, examples of which are described herein.

In order to perform any of the related procedures, the user gains percutaneous access to, for example, the femoral artery, causing a puncture site in the artery. To gain percutaneous access to the artery, the Seldinger technique may be used. For example, a hollow bore needle may be inserted into the artery. A guidewire 150 may then be advanced through the hollow needle and into the femoral artery a sufficient distance to allow removal of the needle without the guidewire 150 pulling out of the vessel. Removing the needle leaves the guidewire 150 in place, with a portion of the guidewire 150 extending into the artery. The guidewire 150, extending from outside the patient into the femoral artery, provides for an entry guide for other medical devices including the vascular closure device 10. Therefore, once the guidewire 150 is positioned in the vessel of the patient, catheters or introducers of gradually increasing diameters may be advanced over the guidewire and through the puncture into the artery to further open the puncture site. Then, an introducer/procedure access sheath set (i.e. an introducer inside an access tube or sheath) may be moved along the guidewire 150 such that a distal end of the sheath moves into the vessel through the puncture site. Once positioned, the introducer can be removed such that the sheath provides for sizable access to the vessel interior from outside the body.

After the relevant procedure is completed, the puncture site in the artery created by the bore needle during percutaneous access of the artery may be closed. The vascular closure device 10 may be used to seal the puncture site. FIGS. 14-23 show schematic views of the vascular closure device 10 during the process of closing a puncture site 200 in a vessel (e.g. artery) wall 204.

Now in reference to FIG. 14, to deliver the vascular closure device 10 to the puncture site 200 so that the closure device 10 can seal the puncture site 200, the introducer/procedure sheath set may be replaced with a closure access sheath 208. For example, as shown in FIG. 14, the procedure sheath may be exchanged for the closure access sheath 208 by removing the procedure sheath from the patient, leaving the guidewire 150 in place, and subsequently moving the closure access sheath 208 along the guidewire 150 or otherwise positioning the access sheath 208, such that a portion of the access sheath 208 is disposed within the vessel through the puncture site 200. As shown in FIG. 14, the access sheath 208 defines a distal end DA, a proximal end PA, and an access channel 212 that extends from the proximal end PA to the distal end DA along an insertion direction I. The access sheath 208 may further include a sheath hub 216 at its proximal end PA. The sheath hub 216 may be configured to couple to the vascular closure device 10 when the vascular closure device 10 is inserted into the access channel 212 along the insertion direction I.

As shown in FIG. 15, the vascular closure device 10 can be positioned by translating, extending, or otherwise advancing the vascular closure device 10 into and through the access channel 212 along the insertion direction I such that a portion of the footplate 40 (e.g., distal portion 41d) protrudes from the distal end DA of the access sheath 208 and into the vessel. Once fully inserted, the vascular closure device 10 can couple to the sheath hub 216. A proximal end of the footplate 40 may be coupled to the release component 22 and the delivery component 26 while the vascular closure device 10 is being moved into the vessel through the puncture site 200 of the vessel. While the proximal end of the footplate 40 is fixed, the footplate 40 may be oriented in a pre-sealing position whereby at least the proximal end of the footplate 40 is concealed and thus prevented from dragging against the vessel wall during positioning of the footplate 40 within the vessel.

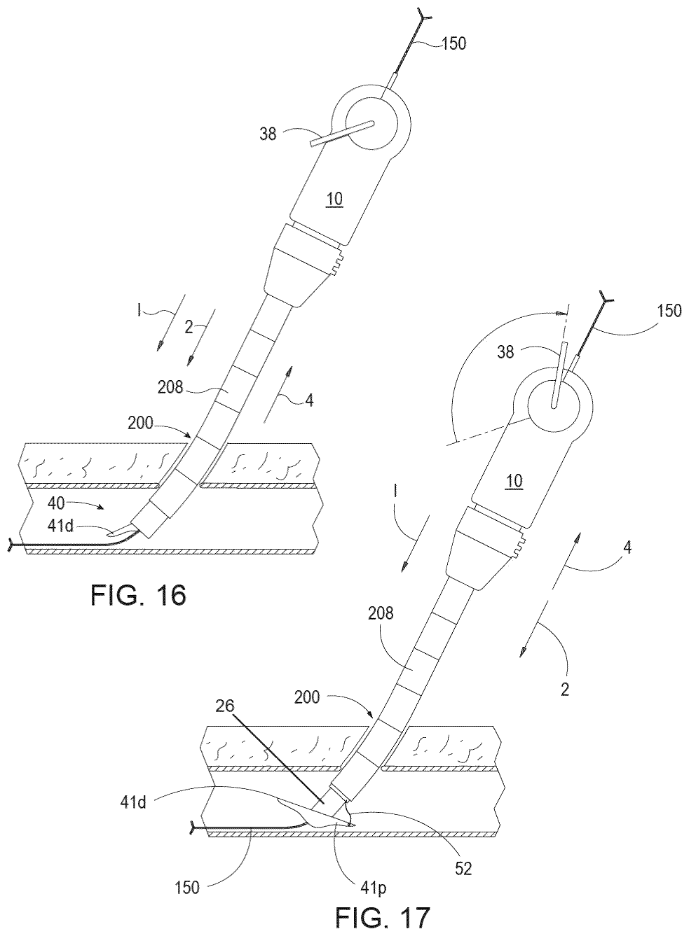

Once the vascular closure device 10 is properly positioned within the access sheath 208, the footplate 40, and in particular, the entire access sheath 208 and vascular closure device 10 combination, can be moved proximally such that the footplate 40 is positioned adjacent to the puncture site 200, as shown in FIGS. 16-18. In some situations, the footplate 40 may not be in the proper position for sealing. In this case, the user may determine a position of the footplate 40 using a variety of visualization techniques or tools, such a puncture locators, radiography, or other means to identify the location of the footplate. The user can then retract the footplate 40 in a proximal direction toward the distal end of the deployment assembly 14 and out of the lumen of the artery or vein. Non-limiting embodiments of footplate 40 retraction are disclosed in the U.S. Patent Provisional Application No. 63/517,979 and titled “VASCULAR CLOSURE DEVICE WITH RETRACTION ASSEMBLY FOR REPOSITIONING A FOOTPLATE,” filed on Aug. 7, 2023, the entire contents of which are incorporated by reference herein.

As shown in FIG. 17, once the footplate 40 is in a proper position, the deployment actuator 38 is actuated (by moving in the direction of the curved arrow) to thereby release the footplate 40 from the release tube and subsequently apply a tension to the suture 44 so as to pull the footplate 40 against the distal end of the delivery component 26.

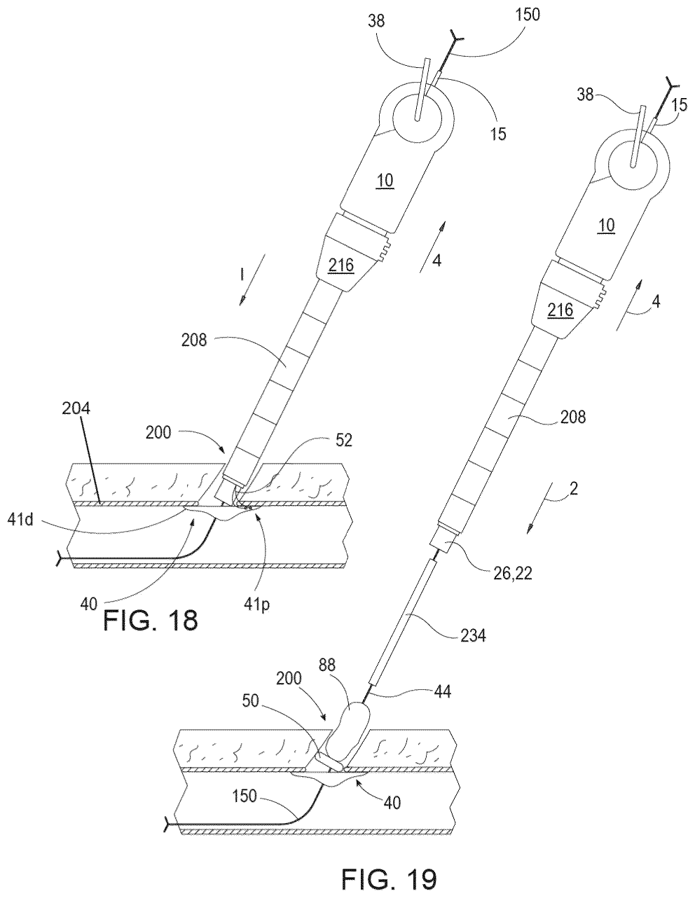

With the footplate 40 in the sealing position shown in FIG. 18, the deployment assembly 14 along with the access sheath 208 can together be pulled proximally such that the footplate 40 abuts the vessel wall 204. As shown in FIG. 19, further pulling of the device 14 and sheath 208 may cause the sealing unit 18, including the footplate 40, plug 88, a locking member 230, suture 44, and a tamper 234, to be fully withdrawn from the delivery component 26. By pulling on the suture 44 in a direction away from the vessel (i.e. in a direction opposite the insertion direction I) the suture 44 may be tensioned and the footplate 40 moved fully into position against an inner surface of the vessel wall 204 at the puncture site 200. The tension in the suture 44 may also pull the plug 88 into the puncture site 200, causing the plug 88 to substantially fill the puncture site 200.

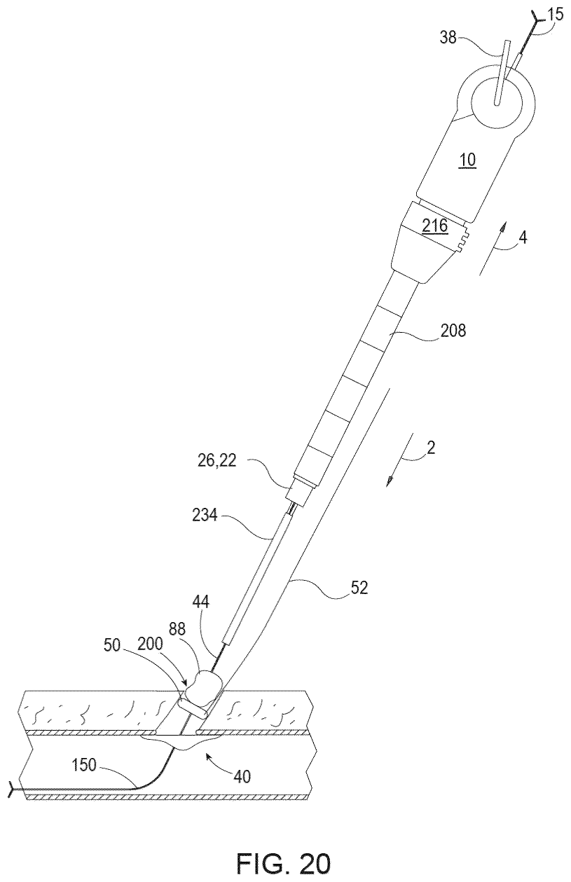

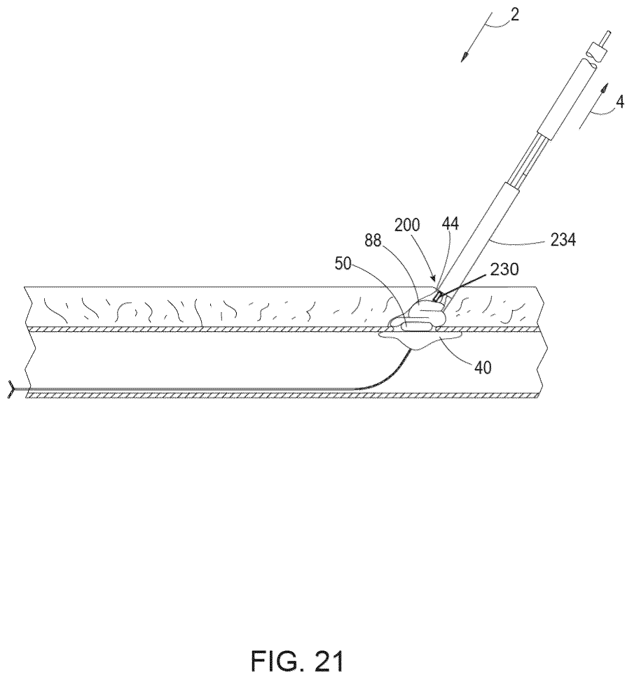

As noted above, in some cases the sealing plug 88 may not be in the proper position for sealing after its insertion. In this case, the user can determine a position of the plug 88 using a variety of visualization techniques, non-limiting examples of which are also noted above. As shown in the progression from FIGS. 19 to 20, the user can retract the scaling plug 88 in a proximal direction toward the distal end of the deployment assembly 14 and out of the lumen of the artery or vein. In this instance, retracting the plug 88 in the proximal direction may be achieved at least in part by retracting a tether 52 coupled to a retraction element 50 that is positioned on the scaling plug 88, for example at a distal end or surface thereof. The sealing plug 88 can be retracted back toward or into the delivery assembly 10 (e.g., delivery component 26). After retracting the plug 88 in the proximal direction using the tether 52 and retraction element 50, the use can readvance the plug 88 in the distal direction toward the lumen of the artery or vein into the correct position, as shown in the progression from FIGS. 20 to 21. After the plug 88 is positioned and in contact with blood or other fluids within the puncture site 200, the plug 88 may expand and fill the remainder of the puncture site 200.

After the user has pulled the suture 44 to cause tension in the suture 44 and to cause the plug 88 to enter the puncture site 200, the user may advance the tamper 234 distally along the guidewire 150 and the suture 44. As shown in FIG. 21, the tamper 234 may contact the locking member 230 and advance the locking member 230 distally along the suture 44 until the locking member 230 contacts the plug 88 and presses the plug 88 against an outer surface of the vessel. As the plug 88 is compressed by the tamper 234, the plug 88 may fold over the top of and inside the puncture site 200. It should be appreciated, however, that in some embodiments, the delivery component 26 may be pulled such that the plug 88 is removed from the delivery component 26 within the release component 22, and the tamper 234 is employed within the release component 22. In such an embodiment, the release component 22 may help control the plug 88 as it is being tamped against the puncture site.

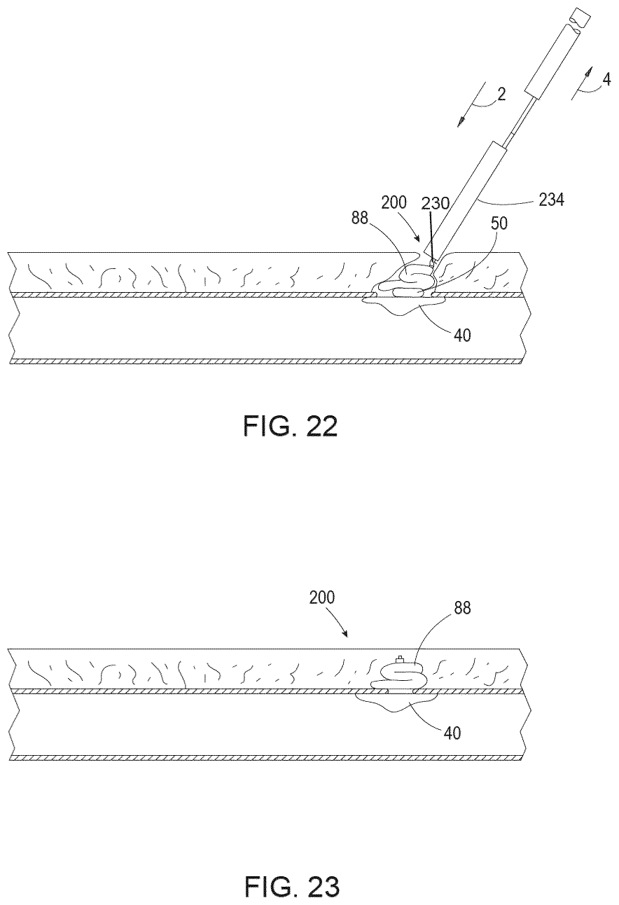

As shown in FIGS. 22 and 23, the locking member 230, together with the plug 88 and the footplate 40, may effect a seal of the puncture site 200. Tension may be maintained on the suture 44 throughout the deployment of the plug 88 from the delivery component 26. After the puncture site 200 is sealed, the guidewire 150 can be removed, as shown in FIG. 22. As the guidewire 150 is removed, the suture 44 may remain in tension, and the user can re-compress the plug 88 with the tamper 234 as desired to confirm a proper seal of the puncture site 200. Once properly sealed, the suture 44 can be cut so that the remaining suture 44, tamper 234, and other components of the sealing unit 18 can be removed from the puncture site 200, as shown in FIG. 23. Remaining portions of the sealing unit 18, including the footplate 40, plug 88, a portion of suture 44, and locking member 230 (depending on material used) may resorb into the body of the patient over time.

While the foregoing description and drawings represent the preferred embodiment of the present invention, it will be understood that various additions, modifications, combinations and/or substitutions may be made therein without departing from the spirit and scope of the present disclosure as defined in the accompanying claims. In particular, it will be clear to those skilled in the art that the present disclosure may be embodied in other specific forms, structures, arrangements, proportions, and with other elements, materials, and components, without departing from the spirit or essential characteristics thereof. One skilled in the art will appreciate that the present disclosure may be used with many modifications of structure, arrangement, proportions, materials, and components, which are particularly adapted to specific environments and operative requirements without departing from the principles of the present disclosure. In addition, features described herein may be used singularly or in combination with other features. For example, features described in connection with one component may be used and/or interchanged with features described in another component. The presently disclosed embodiment is therefore to be considered in all respects as illustrative and not restrictive, the scope of the present disclosure being indicated by the appended claims, and not limited to the foregoing description.

It will be appreciated by those skilled in the art that various modifications and alterations of the present disclosure can be made without departing from the broad scope of the appended claims. Some of these have been discussed above and others will be apparent to those skilled in the art. The following examples provide non-limiting embodiments of various configurations of the devices, assemblies, systems, and methods disclosed herein.

In Example 1, a vascular closure device configured to seal a puncture in an artery or vein may include a deployment assembly, a suture carried by the deployment assembly, and a sealing plug movably coupled to the suture. The sealing plug may have a forward end, a trailing end, and sides that extend from the forward end toward the trailing end. The sealing plug may be carried by the deployment assembly in a first configuration and may be located outside of the deployment assembly in a second configuration. The closure device may also include a retraction element positioned on or at the forward end of the sealing plug. The closure device may also include a tether coupled to the retraction element. When the sealing plug is in the second configuration located outside of the deployment assembly, tension may be applied to the tether, which may pull the retraction element in a proximal direction to move the scaling plug in the proximal direction.

In Example 2, a vascular closure device configured to seal a puncture in an artery or vein may include a suture, a footplate coupled to the suture, a scaling plug movably coupled to the suture, a retraction clement positioned at the sealing plug, and a tether coupled to the retraction element. The footplate may be configured for deployment in a lumen of the artery or vein. The sealing plug may have a forward end, a trailing end, and sides that extend from the forward end toward the trailing end.

In Example 3, a vascular closure device configured to seal a puncture in an artery or vein may include a deployment assembly having a proximal end and a distal end opposite the proximal end. The closure device may also include a suture carried by the deployment assembly, and a footplate carried by the deployment assembly and coupled to the suture. The footplate may be configured to exit the distal end of the deployment assembly for deployment in the puncture. The closure device may also include a sealing plug movably coupled to the suture. The sealing plug may have a forward end, a trailing end, and sides that extend from the forward end toward the trailing end. The sealing plug may be carried by the deployment assembly in a first configuration and may be positioned or located outside of the deployment assembly in a second configuration. The closure device may also include a retraction element positioned at the sealing plug, as well as a tether coupled to the retraction element. When the sealing plug is in the second configuration located outside of the deployment assembly, tension applied to the tether may pull the retraction element in a proximal direction to move the sealing plug in the proximal direction.

In Example 4, the closure device of any one or any combination of Examples 1-3 may be configured such that the retraction element is porous such that blood and other fluids can pass therethrough.

In Example 5, the closure device of any one or any combination of Examples 1-4 may be configured such that the retraction element includes a retraction body having a forward surface and a trailing surface opposite the forward surface, where the trailing surface is opposite a forward end of the sealing plug.

In Example 6, the closure device of any one or any combination of Examples 1-5 may be configured such that the retraction body has a plurality of apertures configured to allow blood or fluid to pass through the retraction element.

In Example 7, the closure device of any one or any combination of Examples 1-6 may be configured such that the retraction element is substantially bioabsorbable.

In Example 8, the closure device of any one or any combination of Examples 1-7 may be configured such that the retraction element is a mesh structure.

In Example 9, the closure device of any one or any combination of Examples 1-4 may be configured such that the retraction element has a first portion positioned at a forward end of the sealing plug and a second portion adjacent to the sides of the sealing plug.

In Example 10, the closure device of any one or any combination of Examples 1-4 may be configured such that the retraction element has a distal wall and a sidewall that extends in a proximal direction relative to the distal end, where the distal wall and the sidewall define an internal space that includes the forward end of the sealing plug.

In Example 11, the closure device of Example 10 may be configured such that the distal wall and the side wall include a plurality of apertures.

In Example 12, the closure device of any one or any combination of Examples 9-11 may be configured such that the retraction element is substantially bioabsorbable.

In Example 13, the closure device of any one or any combination of Examples 9-12 may be configured such that the retraction element is a mesh structure.

In Example 14, the closure device of any one or any combination of Examples 1-13 may be configured such that the retraction element is a first retraction element and further comprising a second retraction element.

In Example 15, the closure device of any one or any combination of Examples 1-14 may be configured such that the tether is a suture.

In Example 16, the closure device of any one or any combination of Examples 1-14 may be configured such that the tether is a filament.

In Example 17, the closure device of any one or any combination of Examples 1-14 may be configured such that the tether is a wire.

In Example 18, the closure device of any one or any combination of Examples 1-14 may be configured such that the tether is an elongate (d) shaft.

In Example 19, the closure device of any one or any combination of Examples 1-18 may be configured such that the tether extends through the deployment assembly.

In Example 20, the closure device of any one or any combination of Examples 1-18 may be configured such that the tether extends alongside the deployment assembly.

In Example 21, the closure device of any one or any combination of Examples 1-20 may be configured such that it further includes a movable lock along the suture, and a tamper slidable along the suture, where the tamper is configured to slide the movable lock into engagement with the sealing plug.

In Example 22, the closure device of any one or any combination of Examples 1-21 may be configured such that the deployment assembly further includes a release member and a delivery member, where either or both of the release member and the delivery member are movable relative to the other to release a footplate.

In Example 23, the closure device of Example 1 may be configured to further include a footplate coupled to the suture, where the footplate is configured for deployment in a lumen of the artery or vein.

In Example 24, the closure device of any one or any combination of Examples 1-3 may be configured such that the retraction element is positioned at the forward end of the sealing plug.

In Example 25, a method for sealing a puncture in an artery or vein involves positioning a distal end of a deployment assembly relative to the puncture of the artery or vein, advancing a plug of a sealing unit along a suture out of the distal end of the deployment assembly toward the puncture of the artery or vein, where the plug is movably coupled to the suture, and retracting the plug in a proximal direction.

In Example 26, the method of Example 25 may be implemented such that advancing the plug of the sealing unit out of the distal end of the deployment assembly toward the puncture of the artery or vein further involves pulling the deployment assembly in a proximal direction to induce tension along the suture, thereby causing the plug to exit out of the distal end of the deployment assembly, and tamping the plug toward the puncture with a tamper slidable along the suture.

In Example 27, the method of one or both of Examples 25 or 26 may further involve retracting the plug back inside the distal end of the deployment assembly.

In Example 28, the method of one or any combination of Examples 25-27 may further involve, after retracting the plug into the distal end of the deployment assembly, causing the plug to exit out the distal end of the deployment assembly and into the puncture of the artery or vein.

In Example 29, the method of Example 25 may further involve advancing a footplate of a sealing unit to exit out the distal end of the deployment assembly and into a lumen of the artery or vein, and retracting the footplate back toward a distal end of the deployment assembly.

In Example 30, the method of Example 29 may further involve determining a position of the sealing unit relative to the lumen of the artery or vein.

In Example 31, the method of Example 29 may further involve retracting the footplate back into the distal end of the deployment assembly.

In Example 32, the method of Example 29 may further involve, after retracting the footplate toward the distal end of the deployment assembly, causing the footplate to exit out the distal end of the deployment assembly and into the lumen of the artery or vein.

In Example 33, the method of any one or any combination of Examples 25-32 may further involve inserting an access sheath into the puncture of the artery or the vein, the access sheath having a proximal end that is positioned outside of the artery or the vein, a distal end that is located in the lumen of the artery or the vein, and a channel that extends from the distal end to the proximal end of the access sheath.

In Example 34, the method of any one or any combination of Examples 25-33 may be implemented such that inserting the distal end of the deployment assembly into the puncture of the artery or vein further involves inserting the deployment assembly into the channel of the access sheath until the distal end of the deployment assembly extends out of the distal end of the access sheath.

In Example 35, the method of any one or any combination of Examples 25-34 may be implemented such that the footplate is restrained by a release component and a delivery component of the deployment assembly, where causing the footplate to exit the distal end of the deployment assembly further comprises moving either or both of the release component and the delivery component relative to each other to release the footplate from the deployment assembly.

In Example 36, the method of Example 25 may further involve retracting the plug toward the distal end of the deployment assembly.

In Example 37. the method of any one or any combination of Examples 25-36 may be implemented using any one or any combination of vascular closure device or components thereof of Examples 1-24.

1. A vascular closure device configured to seal a puncture in an artery or vein, the vascular closure device comprising:
a suture;
a footplate coupled to the suture, wherein the footplate is configured for deployment in a lumen of the artery or vein;
a sealing plug movably coupled to the suture, the sealing plug having a forward end, a trailing end, and sides that extend from the forward end toward the trailing end;
a retraction element positioned at the sealing plug; and
a tether coupled to the retraction element.

2. The vascular closure device of claim 1, wherein the retraction element is porous such that blood and other fluids can pass therethrough.

3. The vascular closure device of claim 1, wherein the retraction element includes a retraction body having a forward surface and a trailing surface opposite the forward surface, wherein the trailing surface is opposite a forward end of the sealing plug.

4. The vascular closure device of claim 3, wherein the retraction body has a plurality of apertures configured to allow blood or fluid to pass through the retraction element.

5. The vascular closure device of claim 1, wherein the retraction element is substantially bioabsorbable.

6. The vascular closure device of claim 1, wherein the retraction element comprises a mesh structure.

7. The vascular closure device of claim 1, wherein the retraction element has a first portion positioned at a forward end of the sealing plug and a second portion adjacent to the sides of the sealing plug.

8. The vascular closure device of claim 7, wherein the retraction element has a distal wall and a sidewall that extends in a proximal direction relative to the distal end, wherein the distal wall and the sidewall define an internal space that includes the forward end of the sealing plug.

9. The vascular closure device of claim 8, wherein the distal wall and the sidewall of the retraction element include a plurality of apertures.

10. The vascular closure device of claim 9, wherein the retraction element is substantially bioabsorbable.

11. The vascular closure device of claim 9, wherein the retraction element comprises a mesh structure.

12. The vascular closure device of claim 1, wherein the retraction element is a first retraction element and further comprising a second retraction element.

13. The vascular closure device of claim 1, wherein the tether comprises a suture.

14. The vascular closure device of claim 13, wherein the tether is a filament.

15. The vascular closure device of claim 1, wherein the tether comprises a wire.

16. The vascular closure device of claim 1, wherein the tether comprises an elongated shaft.

17. The vascular closure device of claim 1, wherein the tether extends through the deployment assembly.

18. The vascular closure device of claim 1, wherein the tether extends alongside the deployment assembly.

19. The vascular closure device of claim 1, further comprising a movable lock positioned along the suture, and a tamper slidable along the suture, the tamper configured to slide the movable lock into engagement with the sealing plug.

20. The vascular closure device according to claim 1, further comprising a footplate coupled to the suture, wherein the footplate is configured for deployment in a lumen of the artery or vein.
